(19) United States
(12) Patent Application Publication
(10) Pub. No.: US 2025/0304520 A1
(43) Pub. Date: Oct. 2, 2025

(54) CHEMO-MECHANCIAL RECYCLING SYSTEM & METHOD FOR POST-CONSUMER, METALIZED MULTI-LAYERED PLASTIC PACKAGING (MLP)

(71) Applicant: ASHAYA WASTE RECYCLERS PRIVATE LIMITED, Pune, Maharashtra (IN)

(72) Inventors: Jitendra SAMDANI, Pune (IN); Anish MALPANI, Pune (IN)

(73) Assignee: ASHAYA WASTE RECYCLERS PRIVATE LIMITED, Pune, Maharashtra (IN)

(21) Appl. No.: 18/865,330

(22) PCT Filed: Apr. 24, 2023
(86) PCT No.: PCT/IN2023/050398

§ 371 Date: Nov. 12, 2024

(30) Foreign Application Priority Data
May 12, 2022 (IN) 202221027479

(51) Int. Cl. C07C 51/09; B09B 3/35; B09B 3/70; B09B 3/80; B09B 101/75; C07C 63/26

(52) U.S. Cl. CPC C07C 51/09; B09B 3/35; B09B 3/70; B09B 3/80; C07C 63/26; B09B 2101/75

(57) ABSTRACT

The present invention relates to a recycling system & method. The present invention particularly relates to a chemo-mechanical recycling system & method for post-consumer, metalized multi-layered plastic packaging (MLP) comprising: a washing chamber (I) for washing waste materials sourced from local waste-pickers, a dryer (II) for drying washed waste material, an agglomerator shredder (III) for shredding waste material, a chemical reactor (IV) for simultaneous reaction of de-metallization of MLP and depolymerization of PET within the MLP, a cooling tank for cooling reacted waste material obtained from chemical reactor in a gravity filtration chamber (V) for separating solid and liquid residue, a density segregation chamber for separating polyolefins from metal hydroxide, unreacted PET and cellulose in density, an acidification chamber (VIII) with concentrate sulfuric acid (H2SO4) to obtain white precipitate, a vacuum filtration chamber (IX) to separate white precipitate to obtain terephthalic acid (TPA) as residue which contains sodium sulphate.

### FIELD OF THE INVENTION

The present invention relates to a recycling process. The present invention particularly relates to a chemo-mechanical recycling process for post-consumer, metalized multi-layered plastic packaging (MLP).

### BACKGROUND OF THE INVENTION

Metallized multi-layered plastic packaging (MLP) is used extensively in the food packaging industry to protect food for long periods of time. This is generally used to store snacks such as chips and chocolates.

MLP consist of upto 5 different types of materials including 4-5 different types of plastics polyethylene terephthalate (PET), polyethylene (PE), polypropylene (PP), ethylene-vinyl alcohol (EVOH), polyamide (Nylon, PA), ionomers (EAA, EMAA), and ethylene vinyl acetate (EVA), cellulose (paper), paper board, polymeric materials, metalized layers or aluminium in varying combinations depending on the use case. They are fused together using adhesives or heat, and this composite nature makes it difficult to recycle. Additionally, MLP is very light—it's a high-volume-low-mass material requiring large-volume containers to transport small-mass amounts which makes it expensive and difficult to transport.

For this reason, most MLP ends up either in landfills or on the streets. Globally, only 14% of flexible packaging (like MLP) is collected for recycling and 95% of the plastic packaging material is lost annually and considered very difficult to recycle economically because it's a composite waste,

Conventionally, there are three processes primarily used to recycle MLP:
Waste-to-energy: In this process, MLP is burnt as a source of energy. This generally destroys all the value of the material (a significant downcycle), and if not managed well, can lead to additional air pollution.
Waste-to-fuel: In this process, MLP is anaerobically burnt to produce fuel such as oil/petrol. This is also considered a downcycle as the fuel can only be used once afterwards, and sells at low prices. This method is also energy intensive, and often results in fuel that is contaminated/low grade.
Combined with Cement/Silica: In this process, MLP is combined with silica or cement to forms products such as bricks, furniture, and roads. This is better than landfilling the MLP, but by adding a different type of material to the mix, the new material is arguably even worse for the environment, and not easily recyclable.

Considering the above drawbacks, it is desirable to provide an efficient process for recycling MLP that eliminates the aforesaid drawbacks and provides the upcycle MLP into high-quality materials.

### SUMMARY OF THE INVENTION

The present invention relates to a recycling system and method. The present invention particularly relates to a chemo-mechanical recycling system and method for post-consumer, metalized multi-layered plastic packaging (MLP).

A recycling system for post-consumer, metalized multi-layered plastic packaging (MLP) comprising: a washing chamber I for washing waste materials sourced from local waste-pickers, a dryer II for drying washed waste material; an agglomerator shredder III for shredding waste material; a chemical reactor IV for reacting the MLP with an aqueous solution of NaOH to both simultaneously dissolve the aluminium and depolymerize the PET (that is within the MLP) by alkaline hydrolysis reaction, a cooling tank for cooling reacted waste material obtained from chemical reactor in a gravity filtration chamber V for separating solid and liquid residue, a density segregation chamber VI for separating polyolefins from metal hydroxide, unreacted PET and cellulose based on density, an acidification chamber VIII with concentrated sulfuric acid (H₂SO₄) to obtain white precipitate, a vacuum filtration chamber IX to separate white precipitate to obtain terephthalic acid (TPA) as residue which contains sodium sulphate.

A recycling method for post-consumer, metalized multi-layered plastic packaging (MLP) comprising the steps of: washing by a washing chamber I, waste material sourced from local waste-pickers and waste picker collectives, drying by a dryer II, waste material received after washing, shredding by an agglomerator shredder III, reacting by a chemical reactor IV, the MLP with an aqueous solution of NaOH to simultaneously dissolve the aluminum by converting it into the sodium aluminate or aluminum hydroxide and depolymerizing the PET by alkaline hydrolysis reaction into it's monomer Terephthalic Acid (TPA), while removing odour, food waste, rust, germs and other solid contaminants from the MLP by four-hour “caustic wash” with NaOH solution, cooling in cooling tank, reacted waste material obtained from chemical reactor, separating by a gravity filtration chamber V, solid and liquid materials; collecting by an acidification chamber VIII, filtered residue obtained from the gravity filtration chamber V which contains the disodium terephthalate, separating based on density segregation chamber VI, polyolefins from metal hydroxide, unreacted PET and cellulose, wherein unreacted PET, cellulose and metal hydroxide were stored in chamber and polyolefins were stored in another chamber, acidifying by the acidification chamber VIII disodium terephthalate with concentrate sulfuric acid (H₂SO₄) to obtain white precipitate, separating white precipitate by vacuum filtration chamber IX to get the terephthalic acid (TPA) as residue which contains sodium sulphate, washing obtained TPA with water to get the sodium sulphate free TPA.

One of the objectives of the present invention is to recycle MLP in a cost-effective manner.

Another objective of the present invention is to improve the recyclability of the materials that constitute MLP.

Another objective of the present invention is to provide an environment-friendly recycling process.

Another objective of the present invention is to provide a robust process of recycling.

Another objective of the present invention is to save time.

Another objective of the present invention is to provide a user-friendly recycling method.

### BRIEF DESCRIPTION OF THE DRAWINGS

The foregoing and other objects, features, and advantages of the invention will be apparent from the following detailed description taken in conjunction with the accompanying drawings, wherein:

FIG. 1 illustrates a recycling system for post-consumer, metalized multi-layered plastic packaging (MLP) in accordance with the present invention.

FIG. 2 illustrates a recycling method for post-consumer, metalized multi-layered plastic packaging (MLP) in accordance with the present invention.

### DETAILED DESCRIPTION OF THE INVENTION

Embodiments of the presently disclosed invention will now be described in detail with reference to the drawings wherein like reference numerals designate identical or corresponding elements.

In addition, the singular forms “a,” “an,” and “the” include plural referents unless the context clearly dictates otherwise. In addition, where reference is made to a list of elements (e.g., elements a, b, c), such reference is intended to include any one of the listed elements by itself, any combination of less than all of the listed elements, and/or a combination of all of the listed elements.

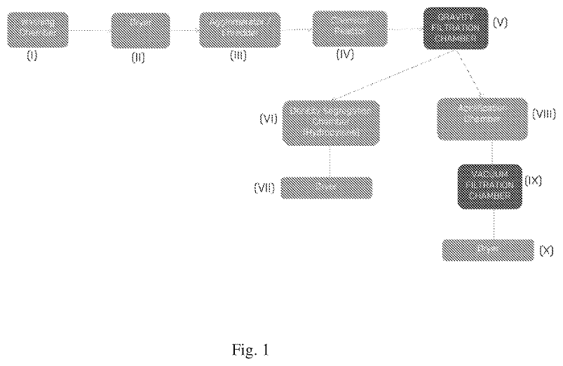

Referring to FIG. 1 illustrate a recycling system for post-consumer, metalized multi-layered plastic packaging (MLP) in accordance with the present invention. The system comprising a washing chamber I where all the waste materials sourced from local waste-pickers and waste picker collectives are placed for washing, after washing the waste material is moved into a dryer II for drying and then moved to an agglomerator or shredder III for shredding. All the waste material was washed to remove the dirt, stain, and oil and dried in oven at 60° C. for almost 20-24 hr, later washed and dried waste material was shredded into 5 mm-10 mm pieces in shredder III.

Further, 5 mm-10 mm pieces of waste material were transferred into the chemical reactor IV with sodium hydroxide (NaOH), phase-transfer catalyst such as tetra butyl ammonium bromide (TBAB) or tetra octy ammonium bromide (TOAB), and water. In chemical reactor IV a simultaneous reaction of demetallization and catalyzed alkaline hydrolysis (of the PET) occur. The catalyst helps to improve the % yield and kinetics of the alkaline hydrolysis without interrupting the de-metallization process (dissolving aluminum using NaOH+Water). The ratio of unsegregated waste material, NaOH, water and catalyst is 1:1:6.7:0.05. This ratio can vary from 1:1:5:0.05-1:1:10:0.05. The reaction is done at 100-120 degrees Celsius for four hours in chemical reactor 18. All these reactions are independently performed without interrupting/stopping each other.

In chemical reactor IV following chemical reactions are occurred simultaneously.
a. It's reacted with an aqueous solution of NaOH (MLP:NaOH in proportions of 1:1) to dissolve the aluminum by converting it into the sodium aluminate or aluminum hydroxide as shown in reaction equation (1) and (2).

6NaOH_{(aq)}+2Alₛ→2Na₃AlO_{3(aq)}+3H_{2(g)} (1)

3NaOH_{(aq)}+Alₛ→Al(OH)₃₍ₛ₎+3Na⁺ (2)
b. Simultaneously, the PET component of MLP (15%-25%) also goes through an alkaline hydrolysis reaction which depolymerizes the PET into it's monomer Terephthalic Acid (TPA). The simple alkaline hydrolysis reaction is shown in reaction equation (3) and (4).

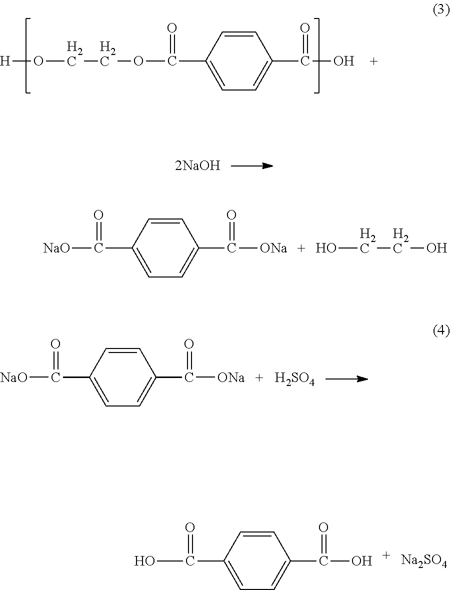

c. Simultaneously the MLP goes through a four-hour “caustic wash” where the NaOH solution thoroughly cleans the MLP waste which removes the odour from the MLP and thoroughly cleans it from food waste, rust, germs and other solid contaminants.

The mixture of reacted waste material and liquid solution obtained from chemical reactor IV is moved towards a cooling tank wherein extra water has been added to cool and dissolve the salt of disodium terephalate, after cooling in the cooling tank the solid and liquid materials was separated by the gravity filtration chamber V.

The solid residue was manually separated and washed again to remove the unseparated disodium terephthalate. Further gravity filtration in gravity filtration chamber V is repeated to get the polyolefins, metal hydroxide, and cellulose in mixer chamber. The filtered obtained from the gravity filtration chamber V were collected in acidification chamber VIII it contains the disodium terephthalate.

The polyolefins were separated from metal hydroxide, unreacted PET and cellulose by using density separation methods in density segregation chamber VI. The mixture of unreacted PET, cellulose and metal hydroxide were stored in chamber, whereas polyolefins were stored in another chamber. The stored polyolefins further used for the extrusion to get 3d-printing filament/granules.

Disodium terephthalate which collected in acidification chamber VIII was acidified with concentrate sulfuric acid (H₂SO₄) and obtained white precipitate. The white precipitate was separated by using vacuum filtration chamber IX to get the terephthalic acid (TPA) as residue. The obtained TPA contains the sodium sulphate, which is washed with water to get the sodium sulphate free TPA. So that the final obtained TPA are used for the polymerization/purification to get the polymerized PET.

The residue obtained from vacuum filtration chamber IX contains acidic and contaminated water which is neutralized with NaOH. Further, neutralized water was tested and ethically disposed in chamber.

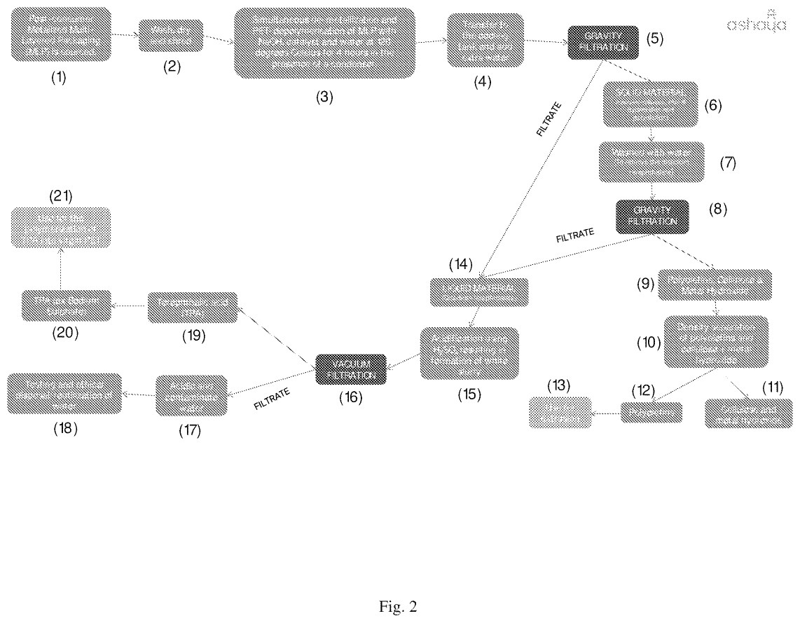

Referring to FIG. 2 illustrate a recycling method for post-consumer, metalized multi-layered plastic packaging (MLP) in accordance with the present invention. The method comprising the steps of, washing waste material sourced from local waste-pickers and waste picker collectives in washing chamber I, drying waste material received after washing in dryer II and then shredding into Agglomerator/shredder III.

After washing, drying and shredding, reacting the MLP with an aqueous solution of NaOH to simultaneously dissolve the aluminum by converting it into the sodium aluminate or aluminum hydroxide and depolymerizing the PET (that's in the MLP) by alkaline hydrolysis reaction into it's monomer Terephthalic Acid (TPA) in the chemical reactor IV, while removing odour, food waste, rust, germs and other solid contaminants from the MLP by four-hour “caustic wash” with NaOH solution.

Cooling reacted waste material obtained from chemical reactor IV in cooling tank, separating solid and liquid materials by the gravity filtration chamber V. Collecting filtered residue obtained from the gravity filtration chamber V in acidification chamber VIII which contains the disodium terephthalate.

Separating polyolefins from metal hydroxide, unreacted PET and cellulose in density segregation chamber VI, wherein unreacted PET, cellulose and metal hydroxide were stored in chamber and whereas polyolefins were stored in another chamber. The stored polyolefins further used for the extrusion to get 3d-printing filament/granules.

Acidifying disodium terephthalate in acidification chamber VIII with concentrate sulfuric acid (H₂SO₄) to obtain white precipitate, separating white precipitate by vacuum filtration chamber IX to get the terephthalic acid (TPA) as residue which contains sodium sulphate, washing obtained TPA with water to get the sodium sulphate free TPA in TPA chamber. So that the final obtained TPA are used for the polymerization/purification to get the repolymerized PET. neutralizing acidic and contaminated water obtained from vacuum filtration chamber IX with NaOH, testing and ethically disposing neutralized water in another chamber.

The kinetics of the alkaline hydrolysis is very slow reaction; hence the present invention used the catalyst to improve the kinetics of the reaction in concentrations of 1%-5% of the total MLP waste. Two catalysts in particular have given good results: tetra butyl ammonium bromide (TBAB) and tetra octyl ammonium bromide (TOAB) which enhances the kinetics by following reaction:

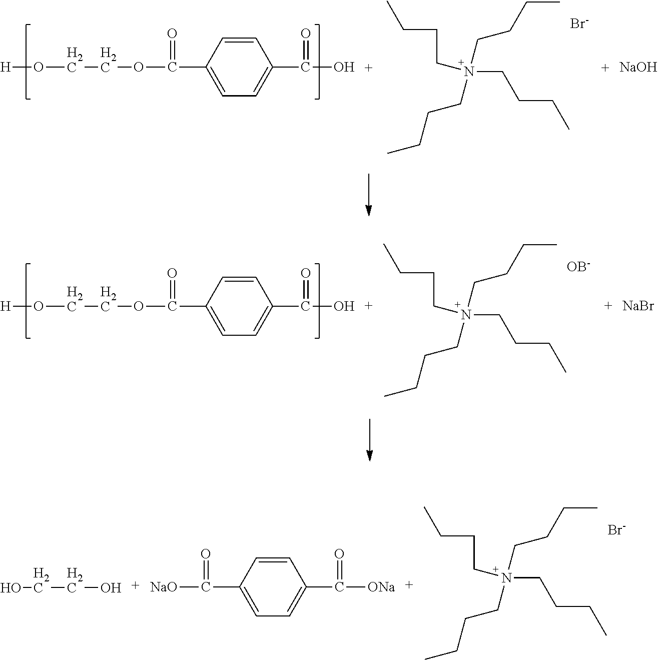

Alternately, the present invention can also use monoethylene glycol (MEG) instead of the phase-transfer catalyst (TBAB/TOAB)+water combination. The ratio of MLP waste to NaOH to MEG is 1:1:10 and does not require an additional catalyst. MEG reacts with NaOH to get the disodium salt of ethylene glycolate as shown in below reaction equation and further it reacts with the PET to get the disodium terephthalate. Disodium terephthalate was acidified with H₂SO₄ in all the cases to get the TPA.

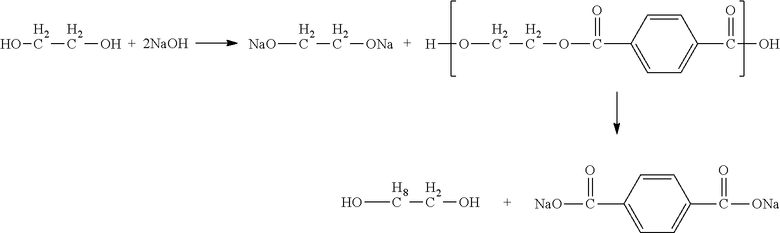

The present invention has been tested on materials from as little as a few grams to batches of 1 kg, from 1 kg batch of MLP, about 90%-95% of materials are recovered for recycling, while also depolymerizing ˜90% of PET into its monomer.

The present invention recovers 85% monoethylene glycol (MEG) after the depolymerization of the PET (using both NaOH+TBAB/TOAB and NaOH+MEG). The other polymer such as polyethylene and polypropylene (polyolefins) do not react with either NaOH or the catalyst so it won't be impacted during the hydrolysis reaction and separation from the PET.

However, during the chemical reaction aluminium can precipitate in the form of aluminium hydroxide and it is present with the polyolefins. Cellulose can also present with polyolefins depending on the contamination levels of the initial waste. To solve for this, we use a conventional density segregation process to separate out the low-density materials (polyolefins) and the high-density materials (cellulose, aluminum hydroxide).

Robustness of the process: The present invention is not limited to recycling MLP but also recycle following types of waste materials and contaminants such as:
PP (polypropylene)
LDPE
HDPE
MDPE
LLDPE
PET polyethylene terephthalate (both coloured and transparent)
PA (Polyamides like PA6 and PA66)*
Polycotton Textile Waste*
Metalized Paper Plates*
Beverage Cartons (like TetraPak)*
Hair/Dust/Tissue Paper*
Polyvinyl Chloride (PVC)*

Present Invention process can handle up to ˜10% of overall contamination by items above that are marked with an asterix.

### The Advantages of Present Invention

Improves recyclability of materials present in MLP: By isolating Polyethylene Terephthalate (PET) and Polyolefins (polypropylene and polyethylene), our process inherently improves the recyclability of the said materials. PET can be infinitely recycled by depolymerization and already has reverse logistics in place as it is one of the most widely used plastics. Polyolefins are less recyclable together, but are a significantly less complex waste than the original MLP (˜6 materials get reduced to only ˜2).
Robust process: This recycling process is robust. It can withstand almost any type of contamination as it separates and removes metal laminates, contaminants, dirt, dust and cellulose. Furthermore, the process can withstand contamination from other types of post-consumer plastic wastes such as PET (#1), HDPE (#2), LDPE (#4) and PP (#5).
Improves economic viability of the process: By consolidating multiple recycling reactions in one-step, the economic feasibility is of the process is improved. This saves time, effort, and overall cost of recycling.

The present invention system & method is therefore much better for the environment than any other current commercial processes.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, from the foregoing description, it will be apparent to one of ordinary skill in the art that many changes and modifications can be made thereto without departing from the spirit or scope of the invention as set forth in the claims.

Accordingly, it is not intended that the scope of the foregoing description be limited to the exact description set forth above, but rather that such description be construed as encompassing such features that reside in the present invention, including all the features and embodiments that would be treated as equivalents thereof by those skilled in the relevant art.

We claim:

1) A recycling system for post-consumer, metalized multi-layered plastic packaging (MLP) comprising:
a washing chamber (I) for washing waste materials sourced from local waste-pickers, a dryer (II) for drying washed waste material;
a agglomerator shredder (III) for shredding waste material;
a chemical reactor (IV) for reacting the MLP with an aqueous solution of NaOH to simultaneously dissolve the aluminium and depolymerize the PET (contained in the MLP) by alkaline hydrolysis reaction in the chemical reactor;
a cooling tank for cooling reacted waste material obtained from the chemical reactor (IV);
a gravity filtration chamber (V) for separating solid and liquid residue,
a density segregation chamber (VI) for separating polyolefins from metal hydroxide, unreacted PET and cellulose in density,
an acidification chamber (VIII) with concentrate sulfuric acid (H₂SO₄) to obtain white precipitate,
a vacuum filtration chamber (IX) to separate white precipitate to obtain terephthalic acid (TPA) as residue which contains sodium sulphate.

2) The recycling system as claimed in claim 1, wherein all the waste material washed in the washing chamber (I) to remove the dirt, stain, and oil and dried in oven at 60° C. for almost 20-24 h, later washed and dried waste material was shredded into 5 mm-10 mm pieces in shredder (III).

3) The recycling system as claimed in claim 2, wherein 5 mm-10 mm pieces of waste material were transferred into chemical reactor (IV) with sodium hydroxide (NaOH), phase-transfer catalyst such as tetra butyl ammonium bromide (TBAB) or tetra octy ammonium bromide (TOAB), and water.

4) The recycling system as claimed in claim 3, wherein the ratio of unsegregated waste material, NaOH, water and catalyst vary from 1:1:5:0.05-1:1:10:0.05. The reaction is done at 100-120 degrees Celsius for four hours.

5) The recycling system as claimed in claim 1, wherein the recycling system is not limited to recycling MLP but also for recycling PP (polypropylene), LDPE, HDPE, MDPE, LLDPE, PET (polyethylene terephthalate-both coloured and transparent), Polycotton Textile Waste, Metalized Paper Plates and Beverage Cartons (like TetraPak).

6) A recycling method for post-consumer, metalized multi-layered plastic packaging (MLP) comprising the steps of:
washing by a washing chamber (I), waste material sourced from local waste-pickers and waste picker collectives;
drying by a dryer (II), waste material received after washing;
shredding by an agglomerator shredder (III);
reacting by a chemical reactor (IV), a simultaneous reaction of de-metallization of MLP and depolymerization of PET within the MLP;
cooling by a cooling tank, reacted waste material obtained from chemical reactor IV;
separating by a gravity filtration chamber (V), solid and liquid residues;
collecting by an acidification chamber (VIII), filtered residue obtained from the gravity filtration chamber (V) which contains the disodium terephthalate.
separating by a density segregation chamber (VI), polyolefins from metal hydroxide, unreacted PET and cellulose, wherein unreacted PET, cellulose and metal hydroxide were stored in chamber and polyolefins were stored in another chamber.
acidifying by the acidification chamber (VIII) disodium terephthalate with concentrate sulfuric acid (H₂SO₄) to obtain white precipitate;
separating white precipitate by a vacuum filtration chamber (IX) to get the terephthalic acid (TPA) as residue which contains sodium sulphate;
washing obtained TPA with water to get the sodium sulphate free TPA in TPA Chamber.

7) The recycling method as claimed in claim 6, wherein the de-metallization of MLP and depolymerization of PET includes reacting the MLP with an aqueous solution of NaOH to simultaneously dissolve the aluminium by converting it into the sodium aluminate or aluminium hydroxide and depolymerize the PET by alkaline hydrolysis reaction into it's monomer Terephthalic Acid (TPA), while removing odour, food waste, rust, germs and other solid contaminants from the MLP by four-hour “caustic wash” with NaOH solution.

8) The recycling method as claimed in claim 6, wherein the solid residue was manually separated and washed again to remove the unseparated disodium terephthalate, the gravity filtration in the gravity filtration chamber (V) was repeated to get the polyolefins, metal hydroxide, and cellulose in a mixer chamber, the filtered residue obtained from the gravity filtration chamber (V) were collected in acidification chamber (VIII), it contains the disodium terephthalate.

9) The recycling method as claimed in claim 6, wherein simultaneous reaction of demetallization and catalyzed alkaline hydrolysis (of the PET) occur without interrupting the de-metallization process, all these reactions are independently performed without interrupting/stopping each other.

10) The recycling method as claimed in claim 6, wherein neutralizing acidic and contaminated water obtained from vacuum filtration chamber IX with NaOH, testing and ethically disposing neutralized water in the chamber.
